(19) United States
(12) Patent Application Publication
(10) Pub. No.: US 2008/0097132 A1
(43) Pub. Date: Apr. 24, 2008

(54) CRACKING OF DICYCLOPENTADIENE

(75) Inventors: John C. Prindle Jr, Baton Rouge, LA (US); Dru J. Manuel, Ventress, LA (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/571,413

(22) PCT Filed: Jun. 30, 2004
(86) PCT No.: PCT/US04/20869

§ 371 Date: Dec. 28, 2006

(51) Int. Cl. C07C 5/31

(52) U.S. Cl. 585/354

(57) ABSTRACT

This invention provides a process for forming monomers from a dimer, wherein the dimer is dicyclopentadiene, di(methylcyclopentadiene), di(ethyleyclopentadiene), or a mixture of any two or more of these. The process comprises i) heating a liquid mixture comprising at least one dimer and at least one liquefying agent in a vaporization zone to at least the vaporization temperature of said mixture to form a vaporized mixture, wherein the vaporization zone consists essentially of a substantially straight conduit in which said liquid mixture occupies less than the entire cross sectional area of said conduit; and ii) introducing at least a portion of the vaporized mixture from i) into a cracking zone such that monomers are formed. This process is conducted at about atmospheric pressure.

### TECHNICAL FIELD

This invention relates to gas-phase thermal cracking of cyclopentadiene dimer to form cyclopentadiene monomer.

### BACKGROUND

Various procedures for cracking cyclopentadiene dimer (dicyclopentadiene) and methylcyclopentadiene dimer, di(methylcyclopentadiene), are known in the art. Some of these procedures crack the dimer in the liquid phase; other procedures crack the dimer in the vapor phase. A problem associated with both liquid-phase cracking and vapor-phase cracking of the cyclopentadiene dimer is severe coking of the heat transfer surfaces, which causes frequent shutdowns for cleaning.

Examples of vapor phase cracking include U.S. Pat. Nos. 3,598,877 and 5,321,177. The process described in U.S. Pat. No. 3,598,877 requires both hydrogen gas and a hydrocarbon to be mixed with the dicyclopentadiene. The hydrogen necessitates the use of superatmospheric pressure in the cracking chamber. In U.S. Pat. 5,321,177, the process described uses water vapor in the cracking process. In some applications of the cyclopentadiene monomer, such as the synthesis of metallocene catalysts, the presence of water is undesirable, and thus the water from the process of U.S. Pat. No. 5,321,177 would need to be removed after the cracking process.

One solution to the above-described problems was a process in which a liquid mixture was formed by mixing at least one dimer and at least one liquefying agent, the liquid mixture was heated in a vaporization zone to at least the vaporization temperature of the mixture to form a vaporized mixture; the vaporized mixture was introduced into a cracking zone such that monomers were formed. Here, the vaporization zone was a coiled tube. Not only was there fouling of the coiled tube, the coiled tube clogged, which was unexpected, and caused frequent shutdowns.

It would be advantageous if a process could be found which effectively cracked the cyclopentadiene dimer, decreased or eliminated fouling of the cracking chamber, and also minimized or eliminated clogging of the vaporization zone. It would be of further advantage if there were a process that produced the monomer in high purity and high yield. An even more desirable cracking process would also not require forcing conditions such as superatmospheric pressure.

### SUMMARY OF INVENTION

This invention enables the achievement of most, if not all, of the above desirable advantages for cracking dicyclopentadiene to form cyclopentadiene; cracking di(methylcyclopentadiene) to form methylcyclopentadiene; and cracking di(ethylcyclopentadiene) to form ethylcyclopentadiene. Monomer can be produced in high conversion from the dimer by the process of the invention. It has been found that the presence of a small amount (relative to the dimer) of a low melting point substance with the dimer reduces the mixture's melting point sufficiently to allow pumping of the material at ambient conditions, i.e., without heating, and, by vaporizing the mixture in a separate zone from the cracking zone, fouling of the cracking zone can be significantly decreased, if not eliminated. This decrease in fouling minimizes shutdown times, e.g., for removing coked material from the cracking zone, or for unclogging the vaporization zone. Thus, this invention provides a process that can be practiced economically on a commercial industrial scale.

An embodiment of this invention is a process for forming monomers from a dimer, where the dimer is dicyclopentadiene, di(methylcyclopentadiene), di(ethylcyclopentadiene), or a mixture of any two or more of these. This process is conducted at about atmospheric pressure. The process comprises

i) heating a liquid mixture comprising at least one dimer and at least one liquefying agent in a vaporization zone to at least the vaporization temperature of said mixture to form a vaporized mixture, wherein the vaporization zone consists essentially of a substantially straight conduit in which said liquid mixture occupies less than the entire cross sectional area of said conduit; and

ii) introducing at least a portion of the vaporized mixture from i) into a cracking zone such that monomers are formed.

This and other embodiments and features of this invention will be still further apparent from the ensuing description and appended claims.

### FURTHER DETAILED DESCRIPTION OF THE INVENTION

The dimeric substances which are converted to their respective monomeric forms in the practice of this invention are dicyclopentadiene, di(methylcyclopentadiene), and di(ethylcyclopentadiene). Mixtures of any two or more of these can be used in the practice of the invention; the dimers in the mixture are converted to their respective monomers. Small amounts of other di(alkylcyclopentadiene)s and higher oligomers of alkylcyclopentadienes may also be present with any combination of the three aforementioned dimers. For the dimers having alkyl groups, the alkyl groups may be in any of various positions on the rings relative to the dimer bonds. As is clear to those of skill in the art, the monomers produced by the practice of the invention are cyclopentadiene (from dicyclopentadiene), methylcyclopentadiene (from di(methylcyclopentadiene)), and ethylcyclopentadiene (from di(ethylcyclopentadiene)).

Dicyclopentadiene is sometimes referred to as “cyclopentadiene dimer”; di(methylcyclopentadiene) is sometimes referred to as “methylcyclopentadiene dimer”; and di(ethylcyclopentadiene) is sometimes referred to as “ethylcyclopentadiene dimer”. Another term for dicyclopentadiene is bis(cyclopentadiene); similarly, another term for di(methylcyclopentadiene) is bis(methylcyclopentadiene); also similarly, another term for di(ethylcyclopentadiene) is bis(ethylcyclopentadiene). Di(ethylcyclopentadiene) is also known as diethyldicyclopentadiene. Throughout this document, the word “dimer”, when not otherwise specified, refers to all of the dimers that may be used in the practice of the invention. Similarly, the word “monomer”, when not otherwise specified, refers to all of the monomers produced by the practice of this invention.

A feature of this invention is the separation of the vaporization zone from the cracking zone. The separation is a temperature separation, in the sense that the temperature in the vaporization zone is at least about 50° C. lower than the temperature in the cracking zone. Without wishing to be bound by theory, it is believed that separation of the vaporization and cracking zones results in the significant decrease in fouling of the cracking zone which is observed in the practice of this invention. Also without wishing to be bound by theory, it is thought that the use of a vaporization zone consisting essentially of a substantially straight conduit in which said dimer only partially occupies the width of said conduit minimizes or eliminates clogging of the vaporization zone.

A liquid mixture comprising the dimer and at least one liquefying agent can be formed by mixing a liquefying agent with the dimer. The liquefying agent should be at least partially miscible with the dimer. The miscibility need not be very great; enough miscibility to form a liquid mixture is all that is necessary. An advantage and a feature of the invention is that, merely by combination with a suitable liquefying agent, and without the use of heat, a liquid mixture comprising the dimer is formed.

It is preferable that the amount of liquefying agent in the mixture is small; preferably, the amount of liquefying agent is not significantly more than is needed to make a liquid that does not have undesirable properties, such as a high viscosity, which would prevent easy transfer of the liquid mixture. The amount of liquefying agent in the liquid mixture is at least enough to form a liquid at ambient conditions when in combination with the dimer. Generally, less than about 25 wt % of the liquefying agent is needed to form a liquid mixture at temperatures between about 10 and about 30° C. when in combination with the dimer. Preferably, the liquefying agent is no more than about 20 wt % of the liquid mixture. Smaller amounts of liquefying agent are typically needed for liquefying agents with lower melting points. More preferably, the liquefying agent is in the range of about 0.5 to about 20 wt % of the liquid mixture; most preferably, the liquefying agent is in the range of about 0.5 to about 10 wt % of the mixture. Thus, another feature of this invention is the high concentration of the dimer in the liquid mixture (e.g., about 80 to about 99.5 wt % in preferred embodiments).

The liquefying agent has a melting point low enough to decrease the melting point of the resultant mixture so that the mixture is a liquid at ambient conditions. Preferably, the liquefying agent is a substance with a melting point below about 0° C. More preferred liquefying agents have a melting point below about −10° C. Substances that can be liquefying agents include ethers, nitriles, ketones, aldehydes, esters, anhydrides, halohydrocarbons, and hydrocarbons. Mixtures of two or more liquefying agents can be used in the practice of the invention. Examples of ethers that may be liquefying agents include diethyl ether, di-n-propyl ether, diisopropyl ether, di-n-butyl ether, butyl methyl ether, butyl ethyl ether, cyclohexylmethyl ether, tetrahydrofuran, tetrahydropyran, 1,3-dioxane, 1,3-dioxolane, glyme (the dimethyl ether of ethylene glycol), 2-methoxyethyl ether (diglyme), triethylene glycol dimethyl ether (triglyme), tetraethylene glycol dimethyl ether (tetraglyme). Nitriles that can be used as liquefying agents include acetonitrile, propionitrile, hexanenitrile, benzonitrile, and the like. Ketones that are suitable liquefying agents include, but are not limited to, acetone, methyl ethyl ketone, pentanone, hexanone, and heptanone. Aldehydes that can be used as liquefying agents include, but are not limited to, benzaldehyde and tolualdehyde. Esters that may be used as the liquefying agent include ethyl acetate, isopropyl acetate, butyl acetate, amyl acetate, hexyl acetate, ethyl formate, diethyl maleate, benzyl acetate, ethyl benzoate, and the like. Examples of anhydrides suitable for use as liquefying agents include acetic anhydride, propionic anhydride, and butyric anhydride. Halohydrocarbons that may be used as liquefying agents include dichloromethane, bromochloromethane, dibromoethane, trichloromethane, tribromomethane, chloroethane, bromoethane, 1,2-dichloroethane, 1-bromo-2-chloroethane, 1-bromopropane, 2-bromobutane, neopentyl chloride, 1,1-dibromopentane, cyclopentyl bromide, 1,6-dibromohexane, trans-1,2-dichlorocyclohexane, 1-chloroheptane, 1-chlorooctane, chlorobenzene, and the like. Other suitable solvents that can be used as liquefying agents include dimethylformamide, N,N-dimethylacetamide, N-methyl pyrrolidinone, and dimethyl sulfoxide. Saturated, unsaturated, straight chain, branched, cyclic, and aromatic hydrocarbons are all suitable liquefying agents. Suitable hydrocarbons include, but are not limited to, pentane, pentene, cyclopentane, hexane, cyclohexene, methylcyclohexane, heptane, heptene, 2-methylhexane, 2,4-dimethylpentane, octane, cyclooctane, cyclooctene, isooctane, 4-methylheptane, 2,5-dimethylhexane, ethylcyclooctane, nonane, decane, benzene, toluene, xylene, ethylbenzene, amylbenzene, methylethylbenzene, diethylbenzene, mesitylene, 1,2,4-triethylbenzene, and tetrahydronaphthalene. Hydrocarbons, especially aromatic hydrocarbons, are preferred liquefying agents. Also preferred as liquefying agents are hydrocarbons having up to about 12 carbon atoms in the molecule. The higher oligomers of the alkylcyclopentadienes are suitable hydrocarbon liquefying agents. Preferred aromatic hydrocarbons include benzene, toluene, xylene, and ethylbenzene. A particularly preferred aromatic hydrocarbon is toluene.

The process of the invention need not be conducted at superatmospheric pressure. Nor is there any need to utilize subatmospheric pressure to make the process work. Of course, in the practice of this invention, small departures from atmospheric pressure may occur, e.g., due to an inadvertent pressure increase in the cracking zone, but such departures are usually small, on the order of about 1.1 to about 2 atmospheres (about 1.1×10⁵ to about 2.03×10⁵ Pascal).

The absence of oxygen is recommended and preferable in the process of the invention due to the flammability of the dimer and the monomer. Water is also preferably substantially absent from the process, although traces of water are not expected to have an adverse effect on the process. It is preferable to operate the process under anhydrous conditions. Those of skill in the art will recognize that small traces of water may inevitably be present under such conditions, but that such water is adventitious. It is advantageous to perform the process of the invention in the absence of water when the monomer obtained is to be used in an application that requires anhydrous conditions. Preferably, the process is conducted in an inert atmosphere. Helium, argon, nitrogen, and the like are suitable gases for use in providing an inert atmosphere. Nitrogen is a preferred gas.

The vaporization zone, exclusive of inlets and outlets, consists essentially of a substantially straight conduit in which the liquid mixture occupies less than the entire cross sectional area of the conduit. The term “substantially straight” indicates that the vaporization zone may have deviations from straightness, but that bends that make less than about a 60° angle are excluded. Vaporization zones having one or more bends that make less than about a 60° angle are undesirable because such vaporization zones are quite prone to clogging. Occupying the entire cross sectional area of the conduit with liquid dimer mixture is believed to significantly increase the probability of clogging the conduit. In a preferred process according to the invention, a portion of the liquid dimer mixture is in contact with at least a portion of the walls of the vaporization zone, usually as a film, and vapor is in the middle of the vaporization zone, away from the walls. More preferably, the liquid mixture occupies no more than about 50% of the cross sectional area of the conduit; even more preferably, no more than about 25% of the cross sectional area is occupied. Still more preferred is to have no more than about 10% of the cross sectional area is occupied. Having no more than about 5% of the cross sectional area of the conduit occupied by the liquid mixture is especially preferred.

The conduit that forms the vaporization zone may be in any convenient shape, including for example, circular, ovoid, triangular, square, or rectangular. Circular and ovoid shapes are preferred; more preferred are circular conduits. A particularly preferred type of vaporization zone is a film evaporator; in these evaporators, a film of the liquid is on the walls of the conduit, and is directly vaporized therefrom. Film evaporators, as is known in the art, are substantially straight evaporators. Without wishing to be bound by theory, film evaporators are believed to efficiently vaporize the di(alkylcyclopentadienes) away from higher oligomers of alkylcyclopentadienes, which are precursors to coke formation; the oligomers are purged from the system as a liquid stream exiting from the film evaporator. Film evaporators include wiped-film evaporators, thin-film evaporators, falling-film evaporators, and the like. Preferably, the vaporization zone is a wiped-film evaporator or a thin-film evaporator.

In the vaporization zone, the liquid mixture is heated to at least its vaporization temperature. The temperature in the vaporization zone is preferably in the range of about 175° to about 250° C.; more preferred is a vaporization zone temperature in the range of about 175° to about 210° C. The temperature at the exit point of the vaporization zone, which is sometimes called the vaporization outlet, should be high enough to maintain the vaporized mixture in the gaseous state. Preferably, the temperature at the exit point is in the range of about 175° to about 250° C. More preferred is a an exit point temperature in the range of about 175° to about 210° C. To prevent coke formation in the vaporization zone, the temperature in the vaporization zone is generally not above about 300° C.

The vaporized mixture is introduced into the cracking zone. The vaporization zone and the cracking zone are separate. It is possible for the vaporization zone and the cracking zone to be in different sections of the same tubing or pipe, so long as the temperature at the exit point of the vaporization zone is at least about 50° C. lower than the temperature at the inlet of the cracking zone. When there is a distance to be traveled from the vaporization zone to the cracking zone, the travel is normally and preferably through tubing or a section of pipe, the walls of which are maintained above the temperature at the exit point of the vaporization zone. In a preferred method, the cracking zone is positioned above the vaporization zone, using the term “above” in the gravitational sense; this operational mode is sometimes referred to as upward flow mode. In upward flow mode, the vaporized dimer flows upward into the cracking zone. An advantage to operating in upward flow mode is that there is no need to force the vaporized mixture in the desired direction of flow. Further, upward flow minimizes the possibility of conveying liquid mixture into the cracking zone. Of course, the many other advantages of the practice of this invention can be achieved even without the use of upward flow mode.

Dimer is converted to monomer in the cracking zone. The cracking zone can be a section of pipe, a column, tubing, and the like. The presence of packing (material) in the cracking zone is desirable, but not necessary. Preferably, the cracking zone is a column. More preferred as the cracking zone is a column packed with a material such as beads, helices, or rings. Temperatures in the cracking zone are generally in the range of about 300° C. to about 500° C. Preferably, the temperatures in the cracking zone are in the range of about 300° to about 450° C. More preferred in the cracking zone are temperatures in the range of about 325° to about 425° C. The temperature at the exit point of the cracking zone is preferably at least about 300° C.

Often, residence times in the cracking zone can be on the order of several seconds, but departures from this are expected. Residence times will vary with at least the scale of the process, the temperature and the pressure in the cracking zone, and the amount of dimer in the now-vaporized liquid mixture. A balance of these factors is preferably taken into account to determine the optimum residence time for a given system, as is known by those of skill in the art. For example, as the amount of liquefying agent in the mixture is increased, the residence time usually needs to be increased to maintain a similar conversion rate of dimer to monomer. However, longer residence times tend to result in increased coking of the cracking zone.

In a preferred embodiment of the invention, at least a portion of the monomer is separated from at least a portion of the remaining dimer. The liquefying agent may be separated with the dimer, or it may remain with the monomer. Once the vapors from the cracking zone have condensed, methods that are well known in the art for separating liquid components, e.g., distillation, may be used. Another method for separation is partial condensation of the vapors exiting the cracking zone in a series of cooled vessels, the series being comprised of at least two vessels, and effecting at least partial separation of the dimer and/or liquefying agent from the monomer. The dimer, once separated, may be recycled to the vaporization zone, with additional dimer and/or liquefying agent as necessary to have them present in the desired proportions. Any liquefying agent present with the dimer may also be recycled to the vaporization zone. Small amounts of monomer may be present in the mixture recycled to the vaporization zone.

Re-dimerization, as re-formation of the dimer is sometimes called, is quite exothermic in addition to being undesirable for reducing the amount of monomer obtained. The monomer is diluted or kept cold (e.g., at temperatures of about 0° C. or below) to prevent the dimer from re-forming. A combination of dilution and cooling can be employed to minimize reformation of the dimer.

The following examples are presented for purposes of illustration, and are not intended to impose limitations on the scope of this invention.

### EXAMPLE 1

The apparatus for this experiment included both a vaporizer and a cracker. The vaporizer was a 0.15 m² (1.6 sq. ft.) thin-film evaporator. The heat transfer fluid in the jacket of the thin-film evaporator was a mixture of diaryl and triaryl ethers (Dowdtherm G, Dow Chemical Company). The cracker was an electrically heated, 10.2 cm (4-inch) I.D., #304 stainless steel pipe. No internal packing was used. Heat was provided to the cracker by six 1-kilowatt U-shaped heating elements (made of INCOLOY, an iron-nickel-chromium alloy). The cracker and vaporizer were mounted at approximately the same elevation. The cracker contained five #304 stainless steel baffles to guide the vapor flow in a direction perpendicular to the heating elements. The cracker was mounted horizontally and the stainless steel line between the vaporizer and cracker was insulated to prevent condensation. The vaporizer exit temperature range was 168-171° C. The heat input to the cracker was controlled to maintain the cracker outlet temperature from 300-420° C.

Dicyclopentadiene (˜97 wt %) having a purity of 97% was mixed with enough toluene to make a 3-5 wt % solution of toluene in dicyclopentadiene. This mixture was then fed through the vaporizer, and was conveyed by natural convection from the vaporizer through the cracker. The vaporizer was fed at a rate of 9.5 kg/hr (21 lb/hr). The oligomers in the feed were removed from the system in the vaporizer as a nonvolatile liquid stream leaving the bottom of the thin-film evaporator. No fouling of the vaporizer was observed. The vapor exiting from the cracker flowed from the cracker outlet through insulated pipe to a heat exchanger and was condensed. The final product mixture, which contained cyclopentadiene monomer, toluene, and a small amount of dicyclopentadiene, after being condensed, was fed into a container having sufficient tetrahydrofuran (THF) to make the final concentration of the cyclopentadiene about 50 wt % in the THF (to minimize re-dimerization). This container was chilled at −5 to −10° C. The final conversion of dicyclopentadiene to cyclopentadiene was usually greater than 97%. Some of the conditions and results for this Example are summarized in Table 1.

TABLE 1

| Cracker outlet | | Conversion to |
|---|---|---|
| temperature | Dimer^{a} feed rate | monomer^{b,c} |
| 300° C. | 8.44 kg/hr (18.6 lb./hr) | 93.6% |
| 325° C. | 9.53 kg/hr (21.0 lb./hr) | 97.2% |
| 325° C. | 11.8 kg/hr (26.0 lb./hr) | 98.5% |
| 350° C. | 9.53 kg/hr (21.0 lb./hr) | 97.9% |
| 420° C. | 9.53 kg/hr (21.0 lb./hr) | 98.1% |

^{a}“Dimer” is dicyclopentadiene.
^{b}“Monomer” is cyclopentadiene.
^{c}Conversion was determined by NMR.

### COMPARATIVE EXAMPLE 1

In this Example, testing of a dicyclopentadiene/toluene mixture in a vaporizer unit was conducted to determine the degree of fouling. The vaporizer consisted of 0.635-cm (0.25-inch) O.D., 22-gauge stainless steel tubing (54 coils of 4.66 m, 15.3 ft.) total length and a straight section 1.22 m (4.0 ft.) in total length inside a 5.1 cm (2-inch) I.D. carbon steel shell (rated for high temperatures). The vaporizer configuration was countercurrent flow downward through the coiled (spiral) tubing section followed by cocurrent flow upward through the straight tubing section. The total heat transfer surface area within the vaporizer was approximately 0.19 m² (2 ft²). A mixture of polydimethylsiloxane (47V20, Rhodia Inc.) and high-temperature (HT) silicone oils was used as the heat transfer fluid during the test for fouling within the vaporizer unit. The vaporizer inlet pressure was measured by a Bourdon-tube pressure gauge. The jacket-side heat transfer fluid was operated at temperatures in the 190-200° C. range. The presence or the absence of condensate at the vaporizer outlet was verified with a small sight glass in the insulated tubing. The vaporizer outlet temperature was measured by a thermocouple that was inserted into the sight glass. The desired vaporizer outlet temperature was 172-185° C.

A liquid mixture of dicyclopentadiene (95 wt %; 99% pure) and toluene (5 wt %) was continuously fed by a peristaltic pump from a 18.93-liter (5-gallon) stainless steel feed tank to the vaporizer. Once steady-state conditions were achieved, this experiment was run continuously for eight hours. After this first day of operation was completed, the vaporizer was cooled to ambient temperature and purged with nitrogen overnight. During the next day, this experiment was continued for four more hours once steady-state conditions were achieved. The steady-state operating conditions during the vaporizer testing summarized in Table 2.

The vaporized dicyclopentadiene/toluene mixture flowed from the vaporizer outlet through insulated 0.635-cm (0.25-inch) O.D. stainless steel tubing to a glass condenser. An increase in the amount of condensation in the sight glass, an increase in the vaporizer inlet pressure at a constant feed rate, or a decrease in the vaporizer outlet temperature indicated increased fouling of the vaporizer.

The condensed dicyclopentadiene/toluene mixture was collected in a flask and was recycled to the feed tank. Approximately two gallons of this dicyclopentadiene solution was pumped through the system a total of six times (to minimize raw material consumption). After the 12-hr experiment was completed, the vaporizer was cooled to ambient temperature and purged with nitrogen overnight.

The condensed dicyclopentadiene/toluene mixture was clear and colorless after the first pass through the system. The amount of pale yellow color in the dicyclopentadiene solution increased slightly after each successive pass through the system. The amount of condensation in the sight glass increased, the vaporizer inlet pressure increased, and the vaporizer outlet temperature decreased after each pass, particularly after the final two passes. These trends indicated that fouling of the vaporizer increased slightly over time.

TABLE 2

| Operating variable | Value |
|---|---|
| Volumetric flow rate of fluid in jacket | 3.785 L/min (1.0 gal/min; |
| | measured at 25° C.) |
| Jacket inlet temperature | 197° C. |
| Jacket outlet temperature | 192° C. |
| Vaporizer inlet pressure | 1.57 × 10⁵ to 1.84 × 10⁵ Pascal |
| | (8-12 psig) |
| Feed rate of dicyclopentadiene solution | 60 g/min (3.63 kg/hr; 8.0 lb./hr.) |

### COMPARATIVE EXAMPLE 2

The apparatus for this experiment included both a vaporizer and a cracker. The vaporizer used in this experiment was the same as that described in Comparative Example 1, except that the heat transfer fluid in the jacket of the vaporizer was a mixture of diaryl and triaryl ethers (Dowtherm G, Dow Chemical Company).

The cracker was an electrically heated, 5.1 cm (2-inch) I.D., 316-stainless steel pipe that was 0.9144 m (3 feet) in total length. The cracker was packed with 0.48 cm ( 3/16-inch) O.D. stainless steel ball bearings. Heat was provided to the cracker by four 500-watt electrical heating rods. The cracker was placed above the vaporizer and was configured in an upright position for upward flow by convection of the vaporized dicyclopentadiene/toluene mixture from the vaporizer through the cracker. All stainless steel tubing between the vaporizer and cracker was heated and insulated to prevent condensation. The vaporizer exit temperature range and the cracker inlet temperature range both were 172-185° C. The heat input to the cracker was controlled to maintain the cracker outlet temperature from 250-350° C. The wall temperature of the cracker always was less than 450° C. to minimize oligomers and tar formation inside the cracker. The system pressure remained under 2.02×10⁵ Pa (2 psig) at all times and usually was less than 1.01×10⁵ Pa (1 psig).

Dicyclopentadiene (99% pure) was mixed with toluene to make a 3-5 wt % solution of toluene in dicyclopentadiene. This mixture was then fed through the vaporizer, and was conveyed by natural convection from the vaporizer through cracker, from the bottom of the cracker and upward through the cracker. The vaporizer was fed as fast as possible based on pressure drop in the system. Clogging of the vaporizer occurred. It is believed to have been caused by both upset conditions that periodically halted the flow and the higher boiling components that entered with the dicyclopentadiene feed and some oligomers/tars that were formed from cyclopentadiene rings combining in the vaporizer. New tubes had to be made up because an acceptable method for cleaning a clogged vaporizer was never found. The coiled tube had to be changed every 6 to 10 hours due to clogging. The vapor exiting from the cracker flowed from the cracker outlet through insulated 0.635-cm (0.25 inch) O.D. stainless steel tubing to a water shell and tube heat exchanger.

Upon exiting the cracker after the final run, the final product mixture, which contained cyclopentadiene monomer, toluene, and a small amount of dicyclopentadiene, was condensed and fed into a container having sufficient tetrahydrofuran (THF) to make the final concentration of cyclopentadiene about 50 wt % in the THF (to minimize re-dimerization). This container was chilled at about −20° C. by submersion in a dry ice bath. The final conversion of dicyclopentadiene to cyclopentadiene was usually greater than 95%.

It is to be understood that the reactants and components referred to by chemical name or formula anywhere in this document, whether referred to in the singular or plural, are identified as they exist prior to coming into contact with another substance referred to by chemical name or chemical type (e.g., another reactant, a solvent, or etc.). It matters not what preliminary chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical operation or reaction or in forming a mixture to be used in conducting a desired operation or reaction. Also, even though an embodiment may refer to substances, components and/or ingredients in the present tense (“is comprised of”, “comprises”, “is”, etc.), the reference is to the substance, component or ingredient as it existed at the time just before it was first contacted, blended or mixed with one or more other substances, components and/or ingredients in accordance with the present disclosure.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation within the spirit and scope of the appended claims.

1. A process for forming monomers from a dimer, wherein the dimer is dicyclopentadiene, di(methylcyclopentadiene), di(ethylcyclopentadiene), or a mixture of any two or more of these, which process is conducted at about atmospheric pressure, and which process comprises
i) heating a liquid mixture comprising at least one dimer and at least one liquefying agent in a vaporization zone to at least the vaporization temperature of said mixture to form a vaporized mixture, wherein the vaporization zone consists essentially of a substantially straight conduit in which said liquid mixture occupies less than the entire cross sectional area of said conduit; and
ii) introducing at least a portion of the vaporized mixture from i) into a cracking zone such that monomers are formed.

2. A process according to claim 1 wherein water is substantially absent from the process.

3. A process according to claim 1 wherein said liquefying agent is no more than about 25 wt % of the liquid mixture.

4. A process according to claim 1 wherein said liquefying agent is in the range of about 0.5 wt % to about 20 wt % of said liquid mixture.

5. A process according to claim 1 wherein said liquefying agent is in the range of about 0.5 wt % to about 10 wt % of said liquid mixture.

6. A process according to claim 1 wherein the liquefying agent has a melting point below about 0° C.

7. A process according to claim 1 wherein said liquefying agent has a melting point below about −10° C.

8. A process according to claim 1 wherein said liquefying agent is an aromatic hydrocarbon.

9. A process according to claim 8 wherein said aromatic hydrocarbon is benzene, toluene, xylene, or ethylbenzene.

10. A process according to claim 9 wherein said aromatic hydrocarbon is toluene.

11. A process according to claim 1 wherein said liquefying agent is in the range of about 0.5 wt % to about 10 wt % of said liquid mixture, and wherein said liquefying agent is an aromatic hydrocarbon.

12. A process according to claim 11 wherein said aromatic hydrocarbon is benzene, toluene, xylene, or ethylbenzene.

13. A process according to claim 11 wherein water is substantially absent from the process.

14. A process according to claim 1 wherein said liquefying agent is at least one higher oligomer of the alkylcyclopentadienes.

15. A process according to claim 1 wherein said conduit is circular or ovoid.

16. A process according to claim 1 wherein the vaporization zone is a film evaporator.

17. A process according to claim 16 wherein said film evaporator is a wiped-film evaporator or a thin-film evaporator.

18. A process according to claim 11 wherein the vaporization zone is a film evaporator.

19. A process according to claim 18 wherein said film evaporator is a wiped-film evaporator or a thin-film evaporator.

20. A process according to claim 1 wherein the liquid mixture occupies no more than about 50% of the cross sectional of the vaporization zone.

21. A process according to claim 1 wherein the liquid mixture occupies no more than about 25% of the cross sectional area of the vaporization zone.

22. A process according to claim 1 wherein the liquid mixture occupies no more than about 10% of the cross sectional area of the vaporization zone.

23. A process according to claim 1 wherein the liquid mixture occupies no more than about 5% of the cross sectional area of the vaporization zone.

24. A process according to claim 1 wherein the temperature at the exit point of said vaporization zone is in the range of about 175° C. to about 250° C.

25. A process according to claim 1 wherein the temperature at the exit point of said vaporization zone is in the range of about 175° C. to about 210° C.

26. A process according to claim I wherein the vaporization zone and the cracking zone are in different sections of the same tubing or pipe, and wherein the temperature in the vaporization zone is at least about 50° C. lower than the temperature in the cracking zone.

27. A process according to claim 1 wherein said cracking zone is a column packed with a material.

28. A process according to claim 1 wherein said process is conducted in an upward flow mode.

29. A process according to claim 1 wherein the temperature in said cracking zone is in the range of about 300° C. to about 450° C.

30. A process according to claim 1 wherein the dimer is dicyclopentadiene.

31. A process according to claim 30 wherein the liquid mixture occupies no more than about 25% of the cross sectional area of the vaporization zone.

32. A process according to claim 30 wherein said liquefying agent is in the range of about 0.5 wt % to about 20 wt % of said liquid mixture, wherein said liquefying agent is an aromatic hydrocarbon, and wherein the vaporization zone is a film evaporator.

33. A process according to claim 32 wherein said aromatic hydrocarbon is benzene, toluene, xylene, or ethylbenzene.

34. A process according to claim 32 wherein said liquefying agent is in the range of about 0.5 wt % to about 10 wt % of said liquid mixture, and wherein said aromatic hydrocarbon is toluene.

35. A process according to claim 34 wherein water is substantially absent from the process.

36. A process according to claim 32 wherein said film evaporator is a wiped-film evaporator or a thin-film evaporator.

37. A process according to claim 34 wherein the temperature at the exit point of said vaporization zone is in the range of about 175° C. to about 250° C.

38. A process according to claim 30 wherein said liquefying agent is in the range of about 0.5 wt % to about 20 wt % of said liquid mixture, wherein said liquefying agent is at least one higher oligomer of the alkylcyclopentadienes, and wherein the vaporization zone is a film evaporator.

39. A process according to claim 1 wherein the dimer is di(methylcyclopentadiene).

40. A process according to claim 39 wherein the liquid mixture occupies no more than about 25% of the cross sectional area of the vaporization zone.

41. A process according to claim 39 wherein said liquefying agent is in the range of about 0.5 wt % to about 20 wt % of said liquid mixture, wherein said liquefying agent is an aromatic hydrocarbon, and wherein the vaporization zone is a film evaporator.

42. A process according to claim 41 wherein said aromatic hydrocarbon is benzene, toluene, xylene, or ethylbenzene.

43. A process according to claim 41 wherein said liquefying agent is in the range of about 0.5 wt % to about 10 wt % of said liquid mixture, and wherein said aromatic hydrocarbon is toluene.

44. A process according to claim 43 wherein water is substantially absent from the process.

45. A process according to claim 41 wherein said film evaporator is a wiped-film evaporator or a thin-film evaporator.

46. A process according to claim 33 wherein the temperature at the exit point of said vaporization zone is in the range of about 175° C. to about 250° C.

47. A process according to claim 39 wherein said liquefying agent is in the range of about 0.5 wt % to about 20 wt % of said liquid mixture, wherein said liquefying agent is at least one higher oligomer of the alkylcyclopentadienes, and wherein the vaporization zone is a film evaporator.

48. A process according to claim 1 which further comprises separating at least a portion of the monomer from at least a portion of the dimer.

49. A process according to claim 48 wherein said separating is done by partial condensation of the vapors exiting the cracking zone.

50. A process according to claim 48 wherein said portion of the dimer is recycled to the vaporization zone.
